(19) United States
(12) Patent Application Publication
(10) Pub. No.: US 2007/0051373 A1
(43) Pub. Date: Mar. 8, 2007

(54) Disposable vibrating condom ring with internal and external vibration

(75) Inventor: Jerome Lin, Taipei City (TW)

Correspondence Address: BACON & THOMAS, PLLC, 625 SLATERS LANE, FOURTH FLOOR, ALEXANDRIA, VA, 22314, US

(73) Assignee: VICON Healthcare International Inc., Taipei Hsien (TW)

(21) Appl. No.: 11/508,237

(22) Filed: Aug. 23, 2006

(30) Foreign Application Priority Data
Sep. 6, 2005 (TW) 094215343

(51) Int. Cl. H02K 33/00; A61F 6/04

(52) U.S. Cl. 128/844; 310/36

(57) ABSTRACT

A disposable vibrating condom ring with internal and external vibration, which is comprised of circular ring, a flexible resin casing, two rigid plastic casings, cylindrical vibrating means and dumbbell shaped vibrating body,wherein the flexible resin casing is integrally formed onto the upper part of said circular ring, and the cylindrical vibrating means coated by two divided rigid plastic casings is inserted inside of the flexible resin casing along the direction of tangent line to the arc of said circular ring and allowed semi-circular opening at the center of each side of said two rigid plastic casings to form a hole slightly bigger than the radius of the neck of the cylindrical vibrating means and allowed said cylindrical vibrating means fully vibrated and jerked to cause internal vibration. The semi-circular opening at the center of upper portion of said two rigid plastic casings to form a hole slightly bigger than the radius of the neck of said dumbbell shaped vibrating body and allow said dumbbell shaped vibrating body unlimitly vibrated to cause external vibration.

### BACKGROUND OF THE INVENTION

1. Field of the Invention

The present invention relates to an electrically operated condom ring assisting a man's sexual function wearing onto penis, particularly to a disposable vibrating condom ring with internal and external vibration.

2. Description of the Related Art

The number of birth is proposed to be controlled as against a population problem systematically is in late years, the use of a condom is recommended as one of the means.

Furthermore, sudden population increase in Africa, Central and South America is combined with concern to a climate change, and becomes to a big problem recently, the restraint becomes with an international problem.

Therefore, including only one child policy in China, each country lays emphasis on a population restraint policy together, but the use of a condom becomes an important point so that these measures give the effectiveness.

In addition to the population problem in late years in the world, it is expected that the practical use of condom is the most realistic measures as a preventive measures against infectious disease to spread AIDS accompanied with the sexual intercourse in an area relating to these AIDS infecting region.

As thus described use of a condom is recommended broadly, but the promotion is still necessary to have a moderate time to be familiarized with a user without resistance from a feeling of social custom and religion, and the development in spreading of a condom without a feeling of resistance is to be expected for a user.

Though the use of a condom spreads economically in the developed nation, the quality of condom improves remarkably in touching feeling in late years, but there is still a resistance from wearing onto penis. This causes a hindrance factor of its promotion. In particular, generation of sexual function began to be impotent or cases having obstacle in sexual function. These influences are too huge to be expected to have function in assisting sexual function.

The inventor proposed an electric condom ring, particularly to a condom ring having a miniature vibrating motor and a battery to be used on a condom to enhance male sexual function in U.S. Pat. No. 6,907,883, by means of structure of the electric condom ring to tight up at local portion of penis to reinforce and to pertain the function, to reach the assisting reinforcement effect by the vibration of the motor inserted into the condom ring, to deduce the uncomfortable feeling of those using of condoms to make vast promotion. However, the miniature vibrating motor located along the ring only vibrates the penis not to reach the passion function ultimately even though the bulges located onto the ring or its outter side can touch the clitoris.

In the above-mentioned prior art, mainly mechanical support and vibration effect can be obtained by using of the vibration mechanism placed perpendicular to the direction of electric condom ring and the tightening effect of condom ring to improve the impotent function and enhance the auxiliary effect. But this action is not one-sided at all, if it is not accepted by two sides both in psychology and affection effect, it will become to adverse effect, finally not to reach the result for vast promotion of condom.

### SUMMARY OF THE INVENTION

This invention relates to a disposable vibrating condom ring with internal and external vibration, which is comprised of circular ring, a flexible resin casing integrally formed onto the upper part of said circular ring, two rigid plastic casings, cylindrical vibrating means and dumbbell shaped vibrating body, wherein the flexible resin casing is integrally formed onto the upper part of said circular ring, and said cylindrical vibrating means coated by two divided rigid plastic casings is inserted inside of said flexible resin casing along the direction of tangent line to the arc of said circular ring, two protruding objects in a height of about 0.3 cm located onto the upper part of said flexible resin casing.

Said two rigid plastic casings are coated onto the outer side of said cylindrical vibrating means in two divided shape and allow semi-circular opening at the center of each side of said two rigid plastic casings to form a hole slightly bigger than the radius of the neck of said cylindrical vibrating means and allow said cylindrical vibrating means fully vibrated and jerked to cause internal vibration.

Semi-circular opening at the center of upper portion of said two rigid plastic casings to form a hole slightly bigger than the radius of the neck of said dumbbell shaped vibrating body and allow said dumbbell shaped vibrating body unlimitly vibrated to cause external vibration. One side of said dumbbell shaped vibrating body is formed into disk like object with undercut shape and the other side are formed many bulges to be knocked with many bulges formed onto the upper portion of said cylindrical vibrating means to allow said dumbbell shaped vibrating body vigorously vibrated and bounced to cause external vibration.

Many bulges integrally formed onto the upward surface of said two plastic casing and the the downward surface of said cylindrical vibrating body will contact each other to cause internal vibration in the restricted space formed by said two rigid plastic casings, and hence to render the external vibration.

Wearing the disposable vibrating condom ring with internal and external vibration of this invention onto the conventional condom, then switching on by pushing the rod type conductive stripe inside the flexible resin casing that covering the front side of the cylindrical vibrating means to allow motor located eccentrically from the center of the vibrating means body that locating at the upper portion of the circular ring driven to vibration, dumbbell shaped vibrating body vigorously vibrated and bounced to contact said bulges formed onto the surface of said cylindrical vibrating means to cause vibrations and bounces. These vibrations and bounces will directly touch the sensitive C point (clitoris) of female sex organ and the two protruding objects in a height of about 0.3 cm located onto the upper part of said flexible resin casing will slightly jerk to scratch the right and left labium.

The vibrating means body used in this invention is located at the upper portion of the circular ring perpendicular to the circular ring, wherein the circular ring is comprised of flexible silicone resin or TPR (thermoplastic rubber) resin of food grade rich in elasticity. The vibrating means body is integrally formed with a ring of ring diameter about 3˜4 cm into thickness of about 2˜4 mm, equipped with a cylindrical vibrating means inside by assembling motor, battery and conductive stripe in a shape of rod or button type, two divided cup shape rigid resin casing are covered onto the outside of the cylindrical vibrating means and clamped to hold the neck of the cylindrical vibrating means, many bulges inside the surface of the rigid resin casings are formed, furthermore a flexible resin casing (cup shape sheath) is covered onto the outside surface of the rigid resin casing, a flexible resin casing is covered onto the front side of the cylindrical vibrating means, the vibration accompanied with the step difference motion caused by contacting the motor rotation to the inside bulges of the eccentrically located cup like rigid resin casing will transfer to the flexible resin casing.

This invention relates to a disposable vibrating condom ring with internal and external vibration, which is comprised of circular ring, vibrating means body located at the upper portion of the circular ring perpendicular to the circular ring, wherein the circular ring is comprised of flexible silicone resin or TPR (thermoplastic rubber) resin of food grade rich in elasticity.

Said vibrating means body is integrally formed with a ring of ring diameter about 3˜4 cm into thickness of about 2˜4 mm, equipped with a cylindrical vibrating means inside by assembling motor, battery and conductive stripe in a shape of rod or button type, a cup shape rigid resin casing is covered onto the outside of the cylindrical vibrating means and clamped to hold the neck of the cylindrical vibrating means, many bulges inside the surface of the resin casing are formed, furthermore a flexible resin cup shape sheath is covered onto the outside surface of the resin casing, a flexible resin hemisphere is covered onto the front side of the cylindrical vibrating means, the vibration accompanied with the step difference motion caused by contacting the motor rotation to the inside bulges of the eccentrically located cup like rigid resin casing will transfer to those flexible resin hemisphere and two flexible resin forming articles located at two shoulders of the vibrating means body. The battery used is of disposable type after using life about 20˜30 min.

Flexible resin hemisphere article and flexible resin forming article located at two shoulders in front of the vibration mechanism body which is located at the ring of the electrically operated condom ring of this invention can receive the vibration accompanied with the step difference motion caused by the vibrating means body and vibrate to transfer to the mate not giving unpleasant or unhappy feeling for cooperation between the partners, the two partners can deduce the uncomfortable feeling when using this condom ring, elevate the sexual feeling each other and try to promote the condom using without resistance.

The conductive stripe located inside said conventional cylindrical vibrating means adopts pushing rod type or switching button type is up to the operation mode of user. Switching button type will be adopted if swtched on from the upper portion of said cylindrical vibrating means. Pushing rod type will be adopted if swithched on from the lower portion of said cylindrical vibrating means.

The disposable vibrating condom ring with internal and external vibration of this invention, which is comprised of circular ring, a flexible resin casing integrally formed onto the upper part of said circular ring, two rigid plastic casings, cylindrical vibrating means and dumbbell shaped vibrating body, said flexible resin casing is integrally formed onto the upper part of said circular ring, wherein the circular ring made of flexible silicone resin or TPR (thermoplastic rubber) resin of food grade rich in elasticity is integrally formed into stripe shaped ring of ununiform diameter of 2˜4 mm, and its size is just enough to put on the penis (the circular ring is formed into ellipsic shape near circular shape of 3˜4 cm in diameter). The ununiform diameter of stripe shaped ring of said circular ring will have jerk of different amplititude due to vibration of said vibrating means. It is preferable to have a bead of about 0.3 cm in diameter formed onto the opposite side of said ciucular ring relative to said flexible resin casing or a bead of about 0.3 cm in diameter formed at the end of a 1 cm long bent rod-like article of flexible resin, this will slightly jerk to wipe the sensitive zone between anus and perineum under vibration state. The bead of 0.5 cm in diameter in the tip of 1 cm long bent rod-like article also can scratch and caress the sensible region between the rectum and the perineum (p point) to enhance sexual love.

Said two rigid plastic casings are coated onto the outer side of said cylindrical vibrating means in two divided shape and allow semi-circular opening at the center of each side of said two rigid plastic casings to form a hole slightly bigger than the radius of the neck of said cylindrical vibrating means and allow said cylindrical vibrating means fully vibrated and jerked. The rigid plastic casings are formed integrally by ABS (acrylonitrile Butadiene Styrene) rigid plastic. This is not restricted by ABS, other rigid plastic which comply the hygenic safety can be adopted.

Said cylindrical vibrating means and said dumbbell shaped vibrating body used in the disposable vibrating condom ring with internal and external vibration of this invention, wherein said dumbbell shaped vibrating body can be substituted by a mushroom shape cylinder without bulges formed at the outer surface to be integrally formed onto said cylindrical vibrating means.

### BRIEF DESCRIPTION OF THE DRAWINGS

FIG. 1 is an enlarged perspective view of a disposable vibrating condom ring with internal and external vibration in accordance with one embodiment of this invention;

FIG. 2 is a plan view of a disposable vibrating condom ring with internal and external vibration of this invention;

FIG. 3 is a rear views of a disposable vibrating condom ring with internal and external vibration of this invention; and

FIG. 4 is a rear views of a disposable vibrating condom ring with internal and external vibration in accordance with another embodiment of this invention.

### DESCRIPTION OF THE PREFERRED EMBODIMENTS

Preferred embodiments of this invention will now be discussed with reference to the drawings.

### EXAMPLE 1

A disposable vibrating condom ring 1 with internal and external vibration of this invention designed and made by the applicant, which is comprised of circular ring 2, a flexible resin casing 20 integrally formed onto the upper part of said circular ring 2, two rigid plastic casings 3, cylindrical vibrating means body 4 and dumbbell shaped vibrating body 5.

Said circular ring 2 which is integrally formed into slim ring like article of different diameter in a range of 2˜4 mm in ring diameter about 3˜4 cm by flexible silicone resin or TPR (thermoplastic rubber) resin of food grade rich in elasticity.

Said flexible resin casing 20 is integrally formed onto the upper part of said circular ring 2, and said cylindrical vibrating means 41 coated by two divided rigid plastic casings 3 is inserted inside of said flexible resin casing 20 along the direction of tangent line to the arc of said circular ring 2, two protruding objects 13 in a height of about 0.3 cm located onto the upper part of said flexible resin casing 20.

Said two rigid plastic casings 3 are coated onto the outer side of said cylindrical vibrating means 41 in two divided shape and allowed semi-circular opening at the center of each side of said two rigid plastic casings 3 to form a hole slightly bigger than the radius of the neck 41,42 of said cylindrical vibrating means 41 and allowed said cylrindrical vibrating means 41 fully vibrated and jerked to cause internal vibration.

Said vibrating means body 4 (about 4 cm in length, about 1 cm in the biggest outer diameter) which is located at the upper portion of said circular ring 2 perpendicular to said circular ring 2, equipped with a cylindrical vibrating means 41 inside by assembling motor, battery and conductive stripe in a shape of rod or button type, said two rigid resin casing 3 are covered onto the outside of the cylindrical vibrating means 41 and clamped to hold the neck of the cylindrical vibrating means 41, many bulges 10 inside the surface of the rigid resin casings are formed, furthermore a flexible resin casing 20 is covered onto the outside surface of the rigid resin casing 3, a flexible resin casing 20 is covered onto the front side of the cylindrical vibrating means body 4, the vibration accompanied with the step difference motion caused by contacting the motor rotation to the inside bulges of the eccentrically located cup like rigid resin casing 3 will transfer to the flexible resin casing 20.

Said semi-circular opening at the center of upper portion of said two rigid plastic casings 3 to form a hole slightly bigger than the radius of the neck 53 of said dumbbell shaped vibrating body 5 and allow said dumbbell shaped vibrating body 5 unlimitly vibrated to cause external vibration, in which one side of said dumbbell shaped vibrating body 5 is formed into disk like object 51 with undercut shape and the other side (head) 52 are formed many bulges 6 to be knocked with many bulges 7 formed onto the upper portion of said cylindrical vibrating means 4 to allow said dumbbell shaped vibrating body 5 vigorously vibrated and bounced to cause external vibration.

Many bulges 10,9 integrally formed onto the upward surface of said two plastic casing 3 and the the downward surface of said cylindrical vibrating body 4 will contact each other to cause internal vibration in the restricted space formed by said two rigid plastic casings 3, and hence to render the external vibration.

Wearing said disposable vibrating condom ring 1 onto the conventional condom, then switching on by pushing the rod type conductive stripe 8 inside the flexible resin casing 20 that covering the front side of the cylindrical vibrating means 4 to allow motor located eccentrically from the center of said vibrating means 4 that locating at the upper portion of said circular ring 2 driven to vibration,said bulges 6 formed onto the surface of said head 52 of said dumbbell shaped vibrating body 5 vigorously vibrated and bounced to contact said bulges 7 formed onto the surface of said cylindrical vibrating means 4 to cause these vibrations and bounces directly touch the sensitive C point of female sex organ and said two protruding objects 13 in a height of about 0.3 cm located onto the upper part of said flexible resin casing 20 will slightly jerk to scratch the right and left labium.

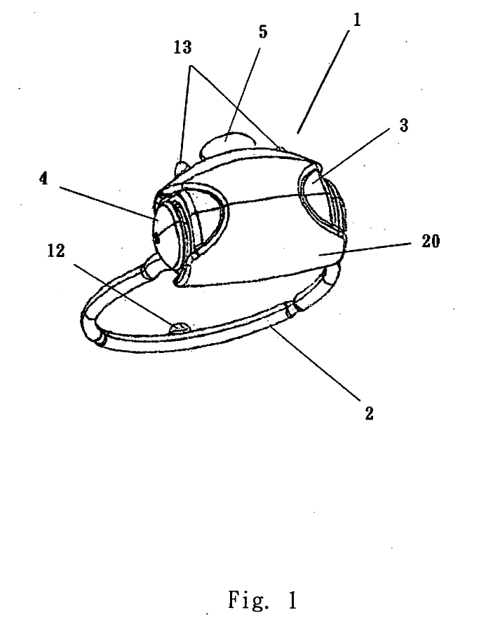

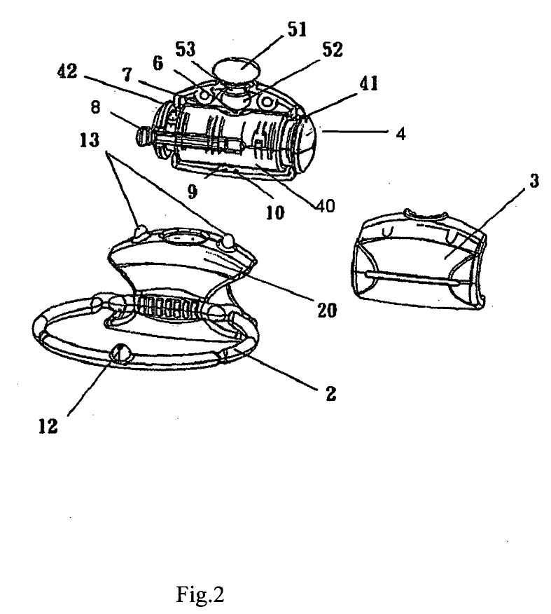

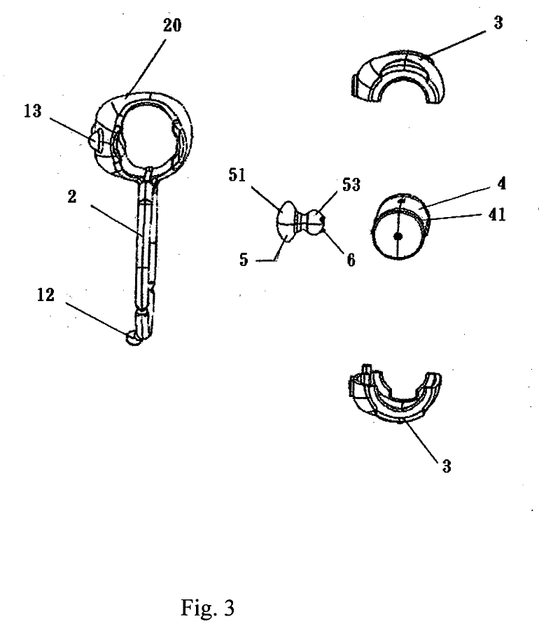

Meanwhile a bead 12 of about 0.3 cm in diameter formed onto the opposite side of said ciucular ring 2 relative to said flexible resin casing 20 or a bead 12 of about 0.3 cm in diameter formed at the end of a 1 cm long bent rod-like article of flexible resin, this will slightly jerk to wipe the sensitive zone between anus and perineum under vibration state. The bead of 0.5 cm in diameter in the tip of 1 cm long bent rod-like article also can scratch and caress the sensible region between the rectum and the perineum to enhance sexual love. (Refer to FIGS. 1, 2 and 3)

A disposable vibrating condom ring 1 with internal and external vibration of this invention can receive the vibration accompanied with the step difference motion caused by the vibrating means body 4 and vibrate to transfer to the mate not giving unpleasant or unhappy feeling for cooperation between the partners, the two partners can deduce the uncomfortable feeling when using this condom ring, elevate the sexual feeling each other and try to promote the condom ring using without resistance.

### EXAMPLE 2

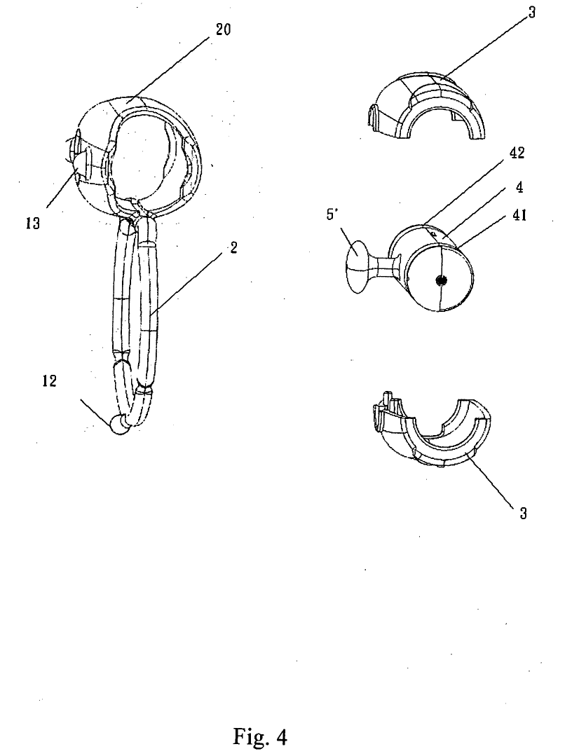

A disposable vibrating condom ring 1 with internal and external vibration of this invention was made according to Example 1 except that said cylindrical vibrating means 4 and dumbbell shaped vibrating body 5 substituted by a rivet (mushroom) shape cylinder 5′ without bulges 6,7,9,10 formed at the outer surface are integrally formed onto said cylindrical vibrating means 4. Said cylindrical vibrating means 4 will make internal vibration in the space restrained by said two rigid resin casing 3 and also make external vibration accompanied with said rivet (mushroom) shape cylinder 5′ to reach the advantageous effect of internal and external vibration of this invention. (Refer to FIG. 4)

What is claimed is:

1. A disposable vibrating condom ring with internal and external vibration, which is comprised of circular ring, a flexible resin casing integrally formed onto the upper part of said circular ring, two rigid plastic casings, cylindrical vibrating means and dumbbell shaped vibrating body, wherein
said flexible resin casing is integrally formed onto the upper part of said circular ring, and said cylindrical vibrating means coated by two divided rigid plastic casings is inserted inside of said flexible resin casing along the direction of tangent line to the arc of said circular ring, two protruding objects in a height of about 0.3 cm located onto the upper part of said flexible resin casing;
said two rigid plastic casings are coated onto the outer side of said cylindrical vibrating means in two divided shape and allowed semi-circular opening at the center of each side of said two rigid plastic casings to form a hole slightly bigger than the radius of the neck of said cylindrical vibrating means and allowed said cylindrical vibrating means fully vibrated and jerked to cause internal vibration;
said semi-circular opening at the center of upper portion of said two rigid plastic casings to form a hole slightly bigger than the radius of the neck of said dumbbell shaped vibrating body and allow said dumbbell shaped vibrating body unlimitly vibrated to cause external vibration, in which one side of said dumbbell shaped vibrating body is formed into disk like object with undercut shape and the other side (head) are formed many bulges to be knocked with many bulges formed onto the upper portion of said cylindrical vibrating means to allow said dumbbell shaped vibrating body vigorously vibrated and bounced to cause external vibration;
many bulges integrally formed onto the upward surface of said two plastic casing and the the downward surface of said cylindrical vibrating body will contact each other to cause internal vibration in the restricted space formed by said two rigid plastic casings, and hence to render the external vibration;
wearing said disposable vibrating condom ring onto the conventional condom, then switching on by pushing the rod type conductive stripe inside the flexible resin casing that covering the front side of the cylindrical vibrating means to allow motor located eccentrically from the center of said vibrating means body that locating at the upper portion of the circular ring driven to vibration, said bulges formed onto the surface of said head of said dumbbell shaped vibrating body vigorously vibrated and bounced to contact said bulges formed onto the surface of said cylindrical vibrating means to cause these vibrations and bounces directly touch the sensitive C point (clitoris) of female sex organ and said two protruding objects in a height of about 0.3 cm located onto the upper part of said flexible resin casing will slightly jerk to scratch the right and left labium.

2. A disposable vibrating condom ring with internal and external vibration as claimed in claim 1 wherein said cylindrical vibrating means and dumbbell shaped vibrating body substituted by a mushroom shape cylinder without bulges formed at the outer surface are integrally formed onto said cylindrical vibrating means.
